(19) United States
(12) Patent Application Publication
(10) Pub. No.: US 2005/0123969 A1
(43) Pub. Date: Jun. 9, 2005

(54) DAP-kinase and HOXA9, two human genes associated with genesis, progression, and aggressiveness of non-small cell lung cancer

(76) Inventors: Chulso Moon, Lutherville, MD (US); Li Mao, Bellaire, TX (US)

Correspondence Address: BANNER & WITCOFF, 1001 G STREET N W, SUITE 1100, WASHINGTON, DC, 20001, US

(21) Appl. No.: 10/983,577

(22) Filed: Nov. 9, 2004

Related U.S. Application Data
(62) Division of application No. 10/045,400, filed on Nov. 29, 2001.
(60) Provisional application No. 60/250,083, filed on Nov. 29, 2000.

(51) Int. Cl. C12Q 1/68; A61K 48/00

(52) U.S. Cl. 435/6; 514/44

(57) ABSTRACT

The invention relates to the discovery of two markers that are informative for one or more of tumorigenesis, tumor progression, and tumor aggressiveness associated with non-small cell lung cancer (NSCLC). The markers are the HOXA9 gene and the gene encoding death-associated protein kinase (DAP-kinase) of humans. Methods of diagnosing NSCLC and methods of assessing the degree of progression and aggressiveness of NSCLC tumors are disclosed, as are methods of inhibiting or alleviating NSCLC. The invention also includes screening methods for identifying compounds that are useful for alleviating, inhibiting, or preventing NSCLC.

### CROSS-REFERENCE TO RELATED APPLICATIONS

This application is entitled to priority, pursuant to 35 U.S.C. § 119(e), to U.S. Provisional Application No. 60/250,083 filed on Nov. 29, 2000.

### STATEMENT REGARDING FEDERALLY SPONSORED RESEARCH OR DEVELOPMENT

This research was supported in part by U.S. Government funds (National Cancer Institute grant number U19 CA68437), and the U.S. Government may therefore have certain rights in the invention.

### REFERENCE TO A MICROFICHE APPENDIX

Not applicable.

### BACKGROUND OF THE INVENTION

Worldwide, lung cancer is by far the most common cause of cancer and cancer related death in men (Parkin et al., 1999, CA Cancer J. Clin. 49:33-64). Lung cancer incidence has also increased significantly in women in recent years (Landis et al., 1998, CA Cancer J. Clin. 48:6-29). Despite improvements in the diagnosis and treatment of this disease in the past two decades, the survival rate remains dismal (Parkin et al., 1999, CA Cancer J. Clin. 49:33-64; Landis et al., 1998, CA Cancer J. Clin. 48:6-29).

Lung cancers can be classified into two major types, non-small cell lung cancer (NSCLC) and small cell lung cancer (SCLC). NSCLC is much more common than SCLC, accounting for about 80% of all lung cancer cases. NSCLC can be divided histologically into two major histologic subtypes: squamous cell carcinoma and adenocarcinoma.

Development of NSCLC is a multi-step process involving accumulation of genetic and epigenetic alterations (Virmani et al., 1998, Genes Chromosomes Cancer 21:308-19; Minna, 1989, Chest 96(Suppl):17S-23S; Thiberville et al., 1995, Cancer Res. 55:5133-5139). Inactivation of tumor-suppressor genes is important in lung tumorigenesis and contributes to abnormal cellular proliferation, transformation, invasion, and metastasis associated with NSCLC (Greenblatt et al., 1994, Cancer Res. 54:4855-4878; Reissmann et al., 1993, Oncogene 8:1913-1919; Rosell et al., 1995, Ann. Oncol. 6 (Suppl 3):S15-S20; Kelley et al., 1995, J. Natl. Cancer Inst. 87:756-761).

For patients afflicted with early-stage NSCLC, standard treatment remains the complete surgical resection of primary tumors. Although this treatment is effective and can cure about 60% of the patients with stage I disease, the remaining 40% of patients will die of the disease within 5 years of surgery (Williams et al., 1981, J. Thorac. Cardiovasc. Surg. 82:70-76). With advances in the early detection of lung cancer (Henschke et al., 1999, Lancet 354:99-105), more patients with lung cancers can be diagnosed at earlier stages, permitting therapeutic or preventive intervention at a clinically relevant time.

The stage at which a lung cancer is detected is not the only determinant of the likelihood of successful treatment or inhibition of the cancer. Some cancers grow and spread (i.e., metastasize) more quickly than others, and are referred to as being more aggressive. Current diagnostic methods cannot accurately identify the aggressiveness of a lung cancer. Thus, the clinician sometime has little basis on which to judge how aggressively a detected tumor should be treated (e.g., whether the tumor should be treated by surgical resection alone, by chemotherapy, by radiation therapy, or by resection coupled with chemotherapy and/or radiation therapy in order to improve long-term survival).

A critical need exists for better diagnostic compositions and methods for classification of early-stage lung cancer. Improved diagnostic ability furthermore would permit analysis of the effectiveness of treatment and screening of potential therapeutic compositions. The present invention satisfies these needs, at least in part, by providing novel informative early stage NSCLC markers.

### BRIEF SUMMARY OF THE INVENTION

The invention relates to a method of diagnosing non-small cell lung cancer (NSCLC) at an early stage in a human. The method comprises assessing expression of the gene encoding DAP-kinase in lung cells of the human (e.g., in cells obtained from the human). A lower degree of expression of the gene in the human, relative to a normal level of expression of the gene in humans not afflicted with NSCLC, is an indication that NSCLC tumorigenesis is occurring in the human. Expression of the gene can be assessed by assessing the methylation state of the gene (or the methylation state of the promoter CpG region of the gene).

The invention also relates to a method of assessing NSCLC tumorigenesis at an early stage in a human. This method comprises assessing methylation of the gene encoding DAP-kinase in lung cells of the human.

The invention includes a method of assessing aggressiveness of a NSCLC tumor in a human. The method comprises assessing methylation of the gene encoding DAP-kinase in lung cells of the human. A higher degree of methylation of the gene an indication that the tumor is more aggressive.

Methods disclosed herein can be used to select among methods of treating a NSCLC tumor in a human, for example by assessing methylation of the gene encoding DAP-kinase in lung cells of the human and selecting a more aggressive treatment when a higher degree of methylation of the gene is detected.

In another aspect, the invention includes a method of inhibiting NSCLC tumorigenesis in a human. This method comprises inhibiting methylation of the DAP-kinase gene in lung cells of the human. Methylation of the DAP-kinase gene in cells of a NSCLC tumor can also be used to inhibit progression of the tumor or to reduce the aggressiveness of the tumor. Alternatively, NSCLC tumorigenesis can be inhibited in a human by de-methylating the DAP-kinase gene in lung cells of the human. This method can also be used to inhibiting progression of a NSCLC tumor or to reduce the aggressiveness of the tumor.

The invention includes a prognostic method of assessing the risk that a human will develop NSCLC. This prognostic method comprises assessing expression of the gene encoding DAP-kinase in lung cells of the human. A lower degree of expression of the gene in the human, relative to a normal level of expression of the gene in humans not afflicted with NSCLC, is an indication that the human is at an increased risk for developing NSCLC.

In still another aspect, the invention includes a method of assessing whether a test compound is useful for one or more of inhibiting NSCLC tumorigenesis, progression of a NSCLC tumor, and aggressiveness of a NSCLC tumor. In this method, methylation of the DAP-kinase gene in the presence of the test compound and methylation of the gene in the absence of the test compound are compared, and a lower degree of gene methylation in the presence of the test compound is an indication that the test compound is useful for the selected purpose.

The invention further includes a method of preventing NSCLC in a human at risk for developing NSCLC by inhibiting methylation of the DAP-kinase gene in lung cells of the human or by enhancing de-methylation of that gene.

The invention includes a method of alleviating NSCLC in a human by inhibiting methylation of the DAP-kinase gene in lung cells of the human or by enhancing de-methylation of that gene.

In another aspect, the invention relates to a method of diagnosing NSCLC at an early stage in a human. This method comprises assessing expression of the HOXA9 gene in lung cells of the human. A greater degree of expression of the gene in the human, relative to a normal level of expression of the gene in humans not afflicted with NSCLC, is an indication that the human is afflicted with NSCLC. This method can also be used to assess the risk that a human will develop NSCLC, a greater degree of expression of the gene in the human, relative to a normal level of expression of the gene in humans not afflicted with NSCLC, being an indication that the human is at an increased risk for developing NSCLC.

NSCLC tumorigenesis can be inhibited in a human by inhibiting expression of the HOXA9 gene in lung cells of the human. Likewise, progression of a NSCLC tumor (i.e., from a lower to a higher diagnostic stage) can be inhibited by inhibiting expression of the HOXA9 gene in cells of the tumor.

The invention includes a screening method for assessing whether a test compound is useful for inhibiting one or both of NSCLC tumorigenesis and progression of a NSCLC tumor. This screening method comprises comparing expression of the HOXA9 gene in the presence of the test compound and expression of the gene in the absence of the test compound. A lower degree of expression in the presence of the test compound is an indication that the test compound is useful for the selected purpose.

The invention further relates to a method of preventing NSCLC in a human at risk for developing NSCLC, the method comprising inhibiting expression of the HOXA9 gene in lung cells of the human.

In another aspect, the invention includes a method of alleviating NSCLC in a human. This method comprising inhibiting expression of the HOXA9 gene in lung cells of the human.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

FIG. 1, comprising FIGS. 1A-1D, is a quartet of graphs which depict the relationship between DAP-kinase hypermethylation in primary NSCLC and probability of survival. The Kaplan-Meier method was used to determine the survival probability and the log-rank test to compare the survival curve between groups. FIG. 1A is a graph depicting overall survival for patients who exhibited the DAP-kinase hypermethylation and patients who did not exhibit the alteration. FIG. 1B is a graph depicting disease-specific survival times for patients exhibited the DAP-kinase hypermethylation and patients who did not exhibit the alteration. FIG. 1C is a graph depicting disease-specific survival times for patients who were afflicted with adenocarcinoma and who exhibited the DAP-kinase hypermethylation and patients who were afflicted with adenocarcinoma and who did not exhibit hypermethylation. FIG. 1D is a graph depicting disease-specific survival times for patients who were afflicted with squamous cell carcinoma and who exhibited the DAP-kinase hypermethylation and patients who were afflicted squamous cell carcinoma and who did not exhibit hypermethylation.

FIG. 2, comprising FIGS. 2A-2F, is a series of images which illustrate the results of assays to detect expression of HOXA9 in cells obtained from patients afflicted with NSCLC. FIG. 2A is an image of results from an assay to detect expression of HOXA9 in primary NSCLC and corresponding normal lung tissues, as assessed by RT-PCR (M indicates DNA size markers; N, indicates normal lung tissues; T indicates primary NSCLC; and Neg, indicates negative control). FIG. 2B-2E are images of results of in situ hybridization experiments to detect HOXA9 gene expression in primary NSCLC (FIG. 2B) and in normal-appearing bronchial epithelium obtained from the same patient (FIG. 2D). In FIGS. 2C and 2E, a sense riboprobe was used to hybridize the same specimens as negative controls. FIG. 2F is an image of the results of an assay to detect HOXA9 expression in bronchial brush specimens obtained from former smokers (P indicates positive control; N indicates negative control; and M indicates DNA size markers).

FIG. 3 is the nucleotide sequence of GENBANK® accession no. X76104.

FIG. 4 is the nucleotide sequence of GENBANK® accession no. NM_{—}002142.

### DETAILED DESCRIPTION OF THE INVENTION

The invention relates to discovery of the involvement of two genes in non-small cell lung cancer (NSCLC), particularly including at the early stages of NSCLC. One of the genes, that encoding death-associated protein kinase (DAP-kinase), has been found to be susceptible to methylation at certain sites, particularly including CpG sites in the 5′-untranslated region of the gene. Methylation of this region inhibits expression of the gene and enhances NSCLC tumorigenesis, tumor progression, and tumor aggressiveness. The other of these two genes, designated HOXA9, is one of the homeobox family of genes, and is expressed beginning at an early stage in the onset of NSCLC. Expression of HOXA9 enhances NSCLC tumorigenesis and tumor progression. The invention includes diagnostic, prognostic, therapeutic, and preventive methods for NSCLC and compositions and kits for use in such methods.

The Gene Encoding DAP-Kinase

In one embodiment, the invention includes a method of diagnosing NSCLC at an early stage in a human. This method comprises assessing expression of the gene encoding DAP-kinase in lung cells of the human. A lower degree of expression of the gene in the human, relative to a normal level of expression of the gene in humans not afflicted with NSCLC, is an indication that NSCLC tumorigenesis is occurring in the human. Expression of this gene is inhibited by methylation in its 5′-untranslated region, presumably by inhibiting translation of the gene.

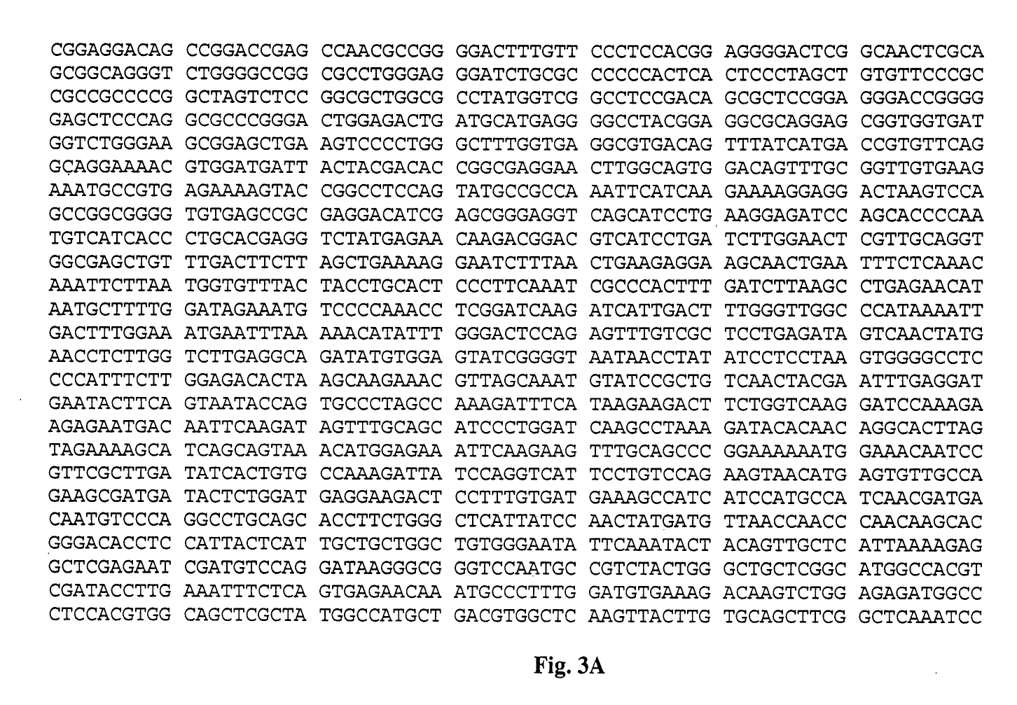

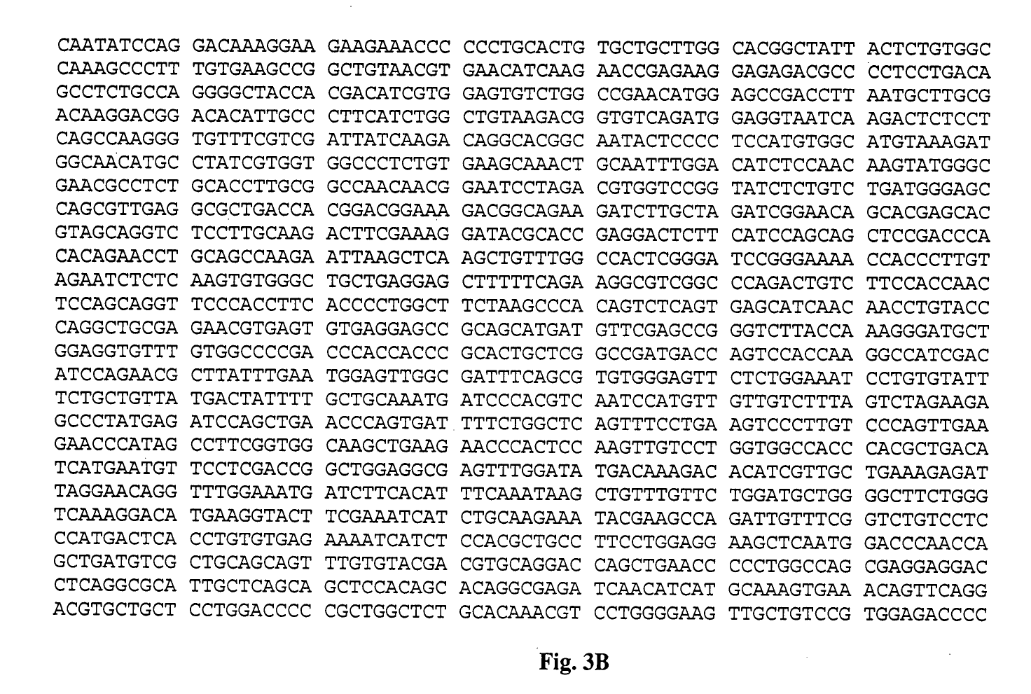

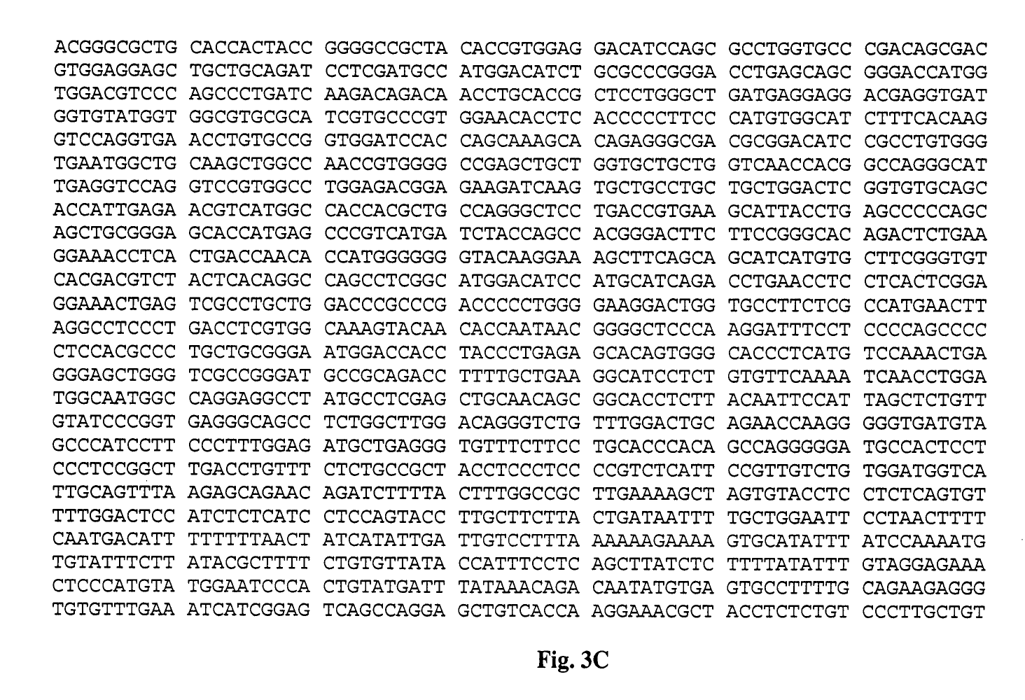

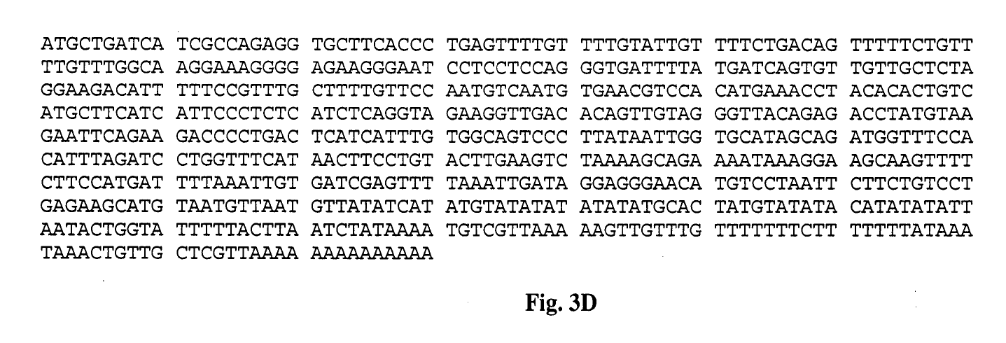

Expression of the gene encoding DAP-kinase (e.g., that corresponding to GENBANK™ accession no. X76104; reproduced in FIG. 3; SEQ ID NO: 4) can be assessed using a variety of known methods. For example, expression of the gene can be assessed in vitro in cells obtained (e.g., by bronchial lavage or biopsy) from a human. Expression of the gene can be assessed directly (e.g., by detecting the primary transcript, the mRNA, or the protein corresponding to the gene) or indirectly, such as by assessing the methylation state of the gene.

A preferred method of assessing the methylation state of the gene comprises assessing the ability of an oligonucleotide to hybridize with the gene in the genome. Alternatively, a pair of oligonucleotide primers able to hybridize with complementary strands of the gene are used, so that a portion of the gene between the two primers can be amplified using known polymerase chain reaction (PCR) procedures. In addition, oligonucleotides or primers which specifically hybridize with a portion of the gene that is susceptible to methylation can be used. In one embodiment, individual oligonucleotides, or oligonucleotide primer pairs, are designed so that the oligonucleotide(s) hybridize with either the methylated or non-methylated form of the complementary region of the gene, but not with both. Using these oligonucleotides, methylated forms of the gene can be differentiated from non-methylated forms, and the methylation state of the gene can be assessed.

Assessment of the methylation state of the gene encoding DAP-kinase in a human (e.g., one who does not exhibit a macroscopic clinical symptom of NSCLC or one afflicted with a diagnostic stage I NSCLC tumor) is informative with respect to i) whether the human is at risk of developing NSCLC; ii) whether the human is afflicted with NSCLC; iii) the degree of progression and likelihood of further progression of NSCLC in the human, and iv) the aggressiveness of an NSCLC tumor in the human. “Aggressiveness” of a tumor refers individually and collectively to the proliferative, invasive, and metastatic prognosis for the tumor. Identification of a tumor as aggressive can indicate that more aggressive therapeutic methods should be employed to treat or inhibit the tumor than might otherwise be employed owing, for example, to side effects and dangers associated with the more aggressive therapy.

Common macroscopic signs and symptoms of NSCLC include a cough that does not go away and gets worse over time, constant chest pain, coughing up blood, shortness of breath, wheezing, or hoarseness, repeated problems with pneumonia or bronchitis, swelling of the neck and face, loss of appetite or weight loss, and fatigue. NSCLC includes various types of lung cancers, including squamous cell carcinoma (i.e., epidermoid carcinoma), adenocarcinoma, large cell carcinoma, adenosquamous carcinoma, and undifferentiated carcinoma.

The methylation state of the gene encoding DAP-kinase can be used in risk assessment methods. In these methods, the methylation state of the gene is assessed in lung cells obtained from a human. A lower degree of expression of the gene in the human, relative to a normal level of expression of the gene in humans not afflicted with NSCLC, is an indication that the human is at an increased risk for developing NSCLC.

Without being bound by any particular theory of operation, it is believed that methylation of the gene encoding DAP-kinase is not only a symptom of NSCLC, but also a contributing factor in NSCLC tumorigenesis, tumor progression, and tumor aggressiveness. Therefore, prevention or inhibition of DAP-kinase gene methylation can inhibit, delay, or prevent one or more of genesis, progression, and aggressiveness of NSCLC tumors. Furthermore, reversal of gene methylation (i.e., enhancement of gene de-methylation) can inhibit or even reverse genesis, progression, and aggressiveness of NSCLC tumors.

Involvement of the gene encoding DAP-kinase in these activities indicates that screening methods that assess the ability of a test compound to inhibit or reverse methylation of the gene can be used to identify compounds useful in treatment, alleviation, or prevention of NSCLC. The invention includes a method of assessing whether a test compound is useful for inhibiting one of i) NSCLC tumorigenesis, ii) progression of a NSCLC tumor, and iii) aggressiveness of a NSCLC tumor. This method comprises comparing methylation of the DAP-kinase gene in the presence of the test compound and methylation of the gene in the absence of the test compound. A lower degree of gene methylation in the presence of the test compound is an indication that the test compound is useful for one or more of these purposes. Once a compound having one of these activities has been identified, it can be incorporated into a pharmaceutical composition suitable for ethical administration to humans and used to alleviate, inhibit, or prevent NSCLC.

The HOXA9 Gene

The invention includes another method of diagnosing NSCLC at an early stage in a human. This method comprising assessing expression of the HOXA9 gene in lung cells of the human. A greater degree of expression of the gene in the human, relative to a normal level of expression of the gene in humans not afflicted with NSCLC, is an indication that the human is afflicted with NSCLC. In fact, expression of the HOXA9 gene in humans not afflicted with NSCLC can be very low or even undetectable. Thus, detection of expression of the HOXA9 gene at all in lung cells (particularly in lung epithelial cells such as those obtained in bronchial lavage, sputum, or biopsy samples) can be indicative of NSCLC in a human. Thus, this method can be used to diagnose NSCLC even in a human who does not exhibit any macroscopic clinical symptom of NSCLC.

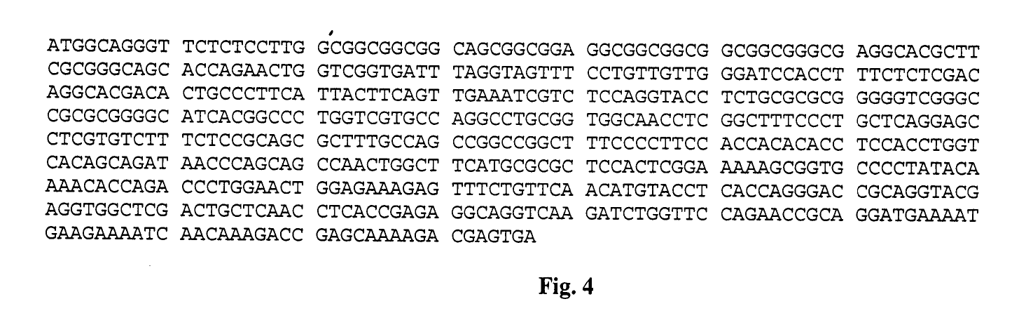

In one embodiment, expression of the HOXA9 gene is assessed using an oligonucleotide that specifically hybridizes with a transcription product of the gene, such as an oligonucleotide described in this disclosure. Because the HOXA9 gene is normally present in the genome of cells, the oligonucleotide preferably does not specifically hybridize with the gene. For example, an oligonucleotide which hybridizes with HOXA9 mRNA (e.g., the mRNA described in GENBANK™ accession no. NM_{—}002142; reproduced in FIG. 4; SEQ ID NO: 6), but not with the HOXA9 gene or its primary transcript can be designed (e.g., by using a sequence which bridges the 3′- and 5′-ends of adjacent exons of the gene). In another embodiment, expression of the gene is assessed using a pair of oligonucleotide primers in a PCR method to amplify a portion of the gene or its corresponding mRNA. For example, the portion can include sub-portions wherein an intron is interposed between the sub-portions in the gene, but wherein the sub-portions are adjacent in mRNA derived from the gene.

Assessment of HOXA9 gene expression can be used to assess the risk that a human will develop NSCLC. In this method, expression of the gene is assessed in lung cells of the human. A greater degree of expression of the gene in the human, relative to a normal level of expression of the gene in humans not afflicted with NSCLC, is an indication that the human is at an increased risk for developing NSCLC.

Without being bound by any particular theory of operation, it is believed that HOXA9 gene expression is not only a symptom of NSCLC, but also a cause of NSCLC tumorigenesis, an enhancer of NSCLC tumor progression, or both. Thus, genesis and progression of NSCLC can be inhibited or prevented by inhibiting or preventing expression of the HOXA9 gene in human lung cells. This can be achieved, for example, by administration of an antisense oligonucleotide (or another composition) designed to inhibit HOXA9 gene transcription, or translation of the mRNA derived therefrom, to human pulmonary epithelial cells.

Involvement of the HOXA9 gene in NSCLC and its onset and progression means that expression of HOXA9 can be used as a marker for assessing the effectiveness of a test compound for alleviating, inhibiting, or preventing NSCLC. The invention includes a method of assessing whether a test compound is useful for inhibiting one of i) NSCLC tumorigenesis and ii) progression of a NSCLC tumor. The method comprises comparing expression of the HOXA9 gene in the presence of the test compound and expression of the gene in the absence of the test compound. A lower degree of expression in the presence of the test compound is an indication that the test compound is useful.

The invention is now described with reference to the following Examples. These Examples are provided for the purpose of illustration only and the invention is not limited to these Examples, but rather encompass all variations which are evident as a result of the teaching provided herein.

### EXAMPLES

### Example 1

Hypermethylation of the DAP-Kinase Promoter Predicts Aggressiveness in Stage I Non-Small Cell Lung Cancer

Death-associated protein kinase (DAP-kinase; also known as DAP-2) is a serine/threonine kinase required for interferon-gamma-induced apoptosis (Feinstein et al., 1995, Genomics 29:305-307). In murine models, lung carcinoma clones which exhibit highly aggressive metastatic behavior lack DAP-kinase expression, and clones which exhibit low metastatic capability express the protein (Inbal et al., 1997, Nature 390:180-184). Restoration of DAP-kinase to physiological levels in highly metastatic carcinoma cells can suppress the metastatic ability of these cells (Inbal et al., 1997, Nature 390:180-184). Thus, association of DAP-kinase expression with metastatic tendency is known, and it can be concluded that DAP-kinase functions, directly or indirectly, as a metastatic suppressor.

Expression of DAP-kinase is repressed in several types of human cancers on account of hypermethylation in the promoter CpG region of the gene (Katzenellenbogen et al., 1999, Blood 93:4347-4353; Kissil et al., 1997, Oncogene 15:403-407; Esteller et al., 1999, Cancer Res. 59:67-70). However, it was not previously known whether decreased expression (or non-expression) of DAP-kinase is associated with early stage NSCLC, or whether decreased expression of this enzyme occurs later in progression of NSCLC. The Experiments presented in this Example were performed in order to determine whether DAP-kinase gene is frequently inactivated by hypermethylation during an early stage of lung tumorigenesis. These experiments also determined whether inactivation of DAP-kinase expression is informative with regard to the aggressiveness of a lung tumor.

In the experiments presented in this Example, surgically resected primary lung tumor tissue samples obtained from 135 patients afflicted with pathologic stage I NSCLC were analyzed in order to determine the methylation status of CpG sites located in the 5′ end of the DAP-kinase gene. Statistical analysis identified the prognostic effect of DAP-kinase gene hypermethylation state on detection of early stage NSCLC and the aggressiveness of the tumor in the patient.

The materials and methods used in the experiments presented in this Example are now described.

Study Population

One hundred and thirty-five patients who had been diagnosed with pathologic stage I NSCLC and had undergone lobectomy or pneumonectomy for complete resection of their primary tumors were enrolled in the study. Patients were followed-up for at least 5 years. The follow-up information was based on chart review and reports from a tumor registry service. None of the patients received adjuvant chemotherapy or radiation therapy before or after surgery. Tissue sections (4 micrometers thick) were obtained from each tissue sample, stained with hematoxylin-eosin, and reviewed by two pathologists to confirm the diagnosis and the presence of tumor cells in the sections.

Microdissection and DNA Extraction

Sections (8 micrometers thick) were obtained from formalin-fixed and paraffin-embedded tissue blocks. Tumorous parts of each section were dissected under a stereomicroscope as described previously (Kim et al., 1997, Cancer Res. 57:400-403; Mao et al., 1996, Nature Med. 2:682-685). Dissected tissues were digested in 200 microliters of digestion buffer containing 50 millimolar Tris-HCl (pH 8.0), 1% (w/v) sodium dodecyl sulfate, and 0.5 milligrams per milliliter proteinase K at 42° C. for 36 hours. The digested products were purified by treating them twice with phenylchloroform. DNA was precipitated using the ethanol precipitation method in the presence of glycogen (obtained from Boehringer-Mannheim, Indianapolis, Ind.) and recovered in distilled water.

Methylation-Specific PCR

Two hundred nanograms of DNA obtained from each tumor sample was used in the initial step of chemical modification. Briefly, DNA was denatured using NaOH and treated with sodium bisulfite (obtained from Sigma, St. Louis, Mo.). After purification using WIZARD™ DNA purification resin (Promega, Madison, Wis.), the DNA was treated again with NaOH. After precipitation, DNA was recovered in water and ready for PCR. PCR was performed using primers which specifically amplified either the methylated DAP-kinase promoter or the non-methylated one, as described (Esteller et al., 1999, Cancer Res. 59:67-70). The primers were the same as those used by Esteller et al.

PCR reactions were performed in a 25-microliter volume containing about 10 nanograms of modified DNA, 3% (v/v) dimethylsulfoxide, 200 micromolar dNTPs, 1.5 millimolar magnesium chloride, 0.4 micromolar PCR primers, and 1.25 units of Taq DNA polymerase (obtained from GIBCO BRL, Gaithersburg, Md.). Amplification was performed for 35 cycles at 95° C. for 30 seconds, 60° C. for 60 seconds, and 70° C. for 60 seconds per cycle, followed by a 5-minute extension at 70° C. in a temperature cycler (HYBAID™, Omnigene, Woodbridge, N.J.) in 500-microliter plastic tubes.

PCR products were separated on 2% (w/v) agarose gels and visualized after staining with ethidium bromide. For each DNA sample, primer pairs specific for methylated DNA and non-methylated DNA were analyzed. Hypermethylation status was determined by visualizing a 98-base pair PCR product using the methylation-specific primer set. All PCR reactions were repeated twice, and the results were reproducible.

Statistical Analysis

Survival probability was computed as a function of time using the Kaplan-Meier estimator. The variance of the Kaplan-Meier estimator was computed by the Greenwood formula. The 5-year survival rates were estimated and compared by the asymptotic Z-test between the hypermethylated and non-hypermethylated groups. The log-rank test was used to compare patient survival times between groups. Both overall survival and disease-specific survival (i.e., death due to lung cancer-related causes) were analyzed. The two-sided chi-squared test was used to test equal proportion between groups in two-way contingency tables. Cox regression was used to model the risks of DAP-kinase hypermethylation on survival time, with adjustment for clinical and histopathological parameters.

The results of the experiments presented in this Example are now described.

A total of 135 patients were evaluated in this study. All patients underwent only surgical treatment for their primary tumors. Ninety-one patients died, and 44 patients were still alive at the time of the last follow-up report. Among the 91 deceased patients, 39 died as a result of lung cancer, 16 as a result of heart diseases, 16 as a result of respiratory diseases, 3 as a result of other organ failures, and 17 for unknown reasons. The median follow-up time was 8.5 years among the surviving patients. Patient ranged in age from 41 to 82 years, with a median age of 62.8 years. Thirty-five (26%) of the patients were women and 100 (74%) were men, which is comparable to the gender distribution of the disease in 1970s and 1980s (Landis et al., 1998, CA Cancer J. Clin. 48:6-29). The probability of 5-year overall survival was 59% and of 5-year disease-specific survival, was 76% in this patient population, similar to probabilities reported in a previous study with a large number of similar patients (Mountain, 1989, Chest 96:47-S49S). The general clinical characteristics of the patients are shown in Table 1.

We analyzed the hypermethylation status of CpG sites located in the 5′-non-translated region of the gene encoding DAP-kinase in primary tumor samples obtained from the 135 patients diagnosed with pathologic stage I NSCLC. Because tumor sections were dissected under a stereomicroscope, tumor cell populations comprised 70 percent or more of most of the specimens. The primer sets for both hypermethylated sequences and non-hypermethylated sequences were tested using non-modified genomic DNA, modified DNA obtained from normal tissues, and modified DNA which exhibited hypermethylation of the CpG sites. DNA was modified as described in Tang et al. (2000, J. Natl. Cancer Inst. 92(18):1460-1461). Non-modified genomic DNA could not be amplified using either the hypermethylated primer set or the non-hypermethylated one. Modified normal and hypermethylated DNA could be effectively amplified using only the corresponding primer sets. Modified DNA from 59 (44 percent) of the 135 tumors could be amplified using the methylation specific primer set and exhibited a specific 98 base pair PCR product, indicating the presence of tumor cells having hypermethylated CpG sites at the critical region of the DAP-kinase gene in these tumors (as indicated in Table 2). Selected PCR amplification products obtained using methylated and non-methylated primer sets were directly sequenced, and the methylation status was verified.

The methylation state of the DAP-kinase gene determined in the tumor samples was analyzed in view of patients' gender and age. No statistical association could be detected between these factors, although there was a trend toward more frequent methylation in men (P=0.09). Hypermethylation was observed more frequently in adenocarcinoma and other histologic types (large cell and unclassified tumors) than it was in squamous cell carcinoma (P=0.02, as indicated in Table 2).

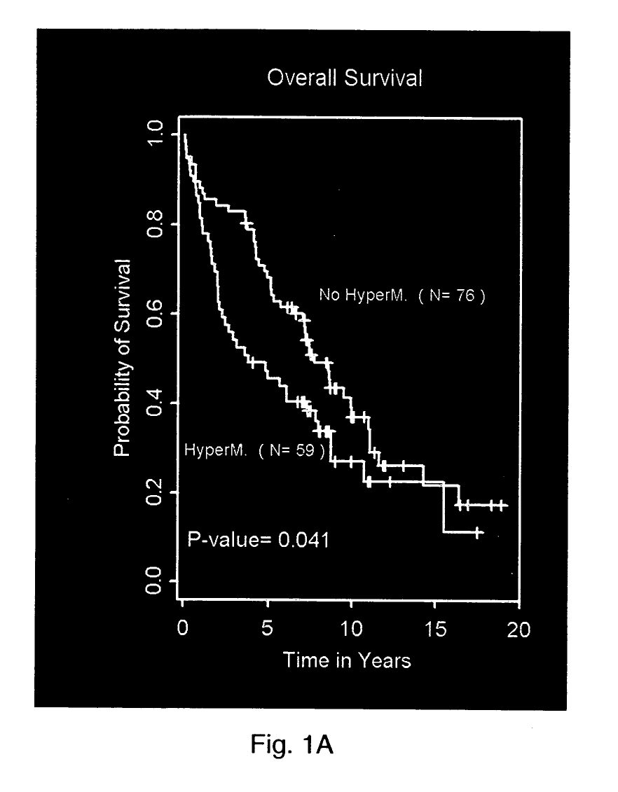

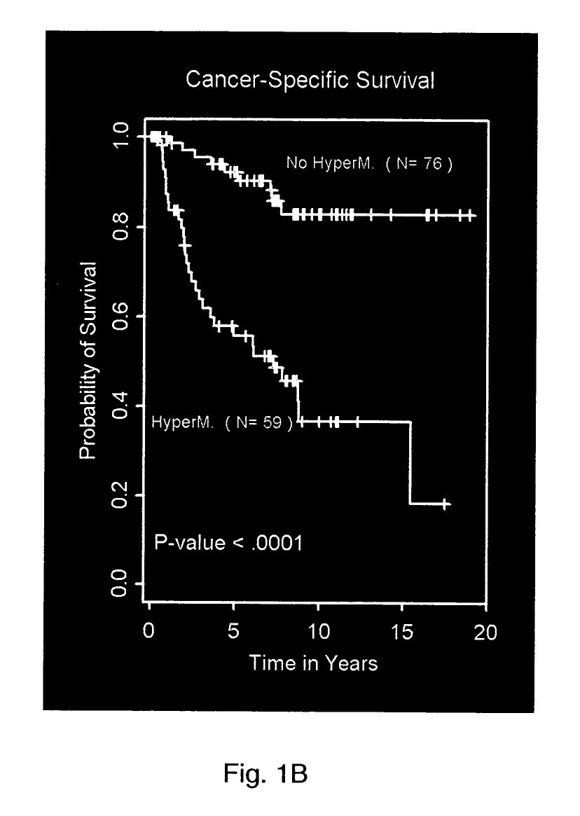

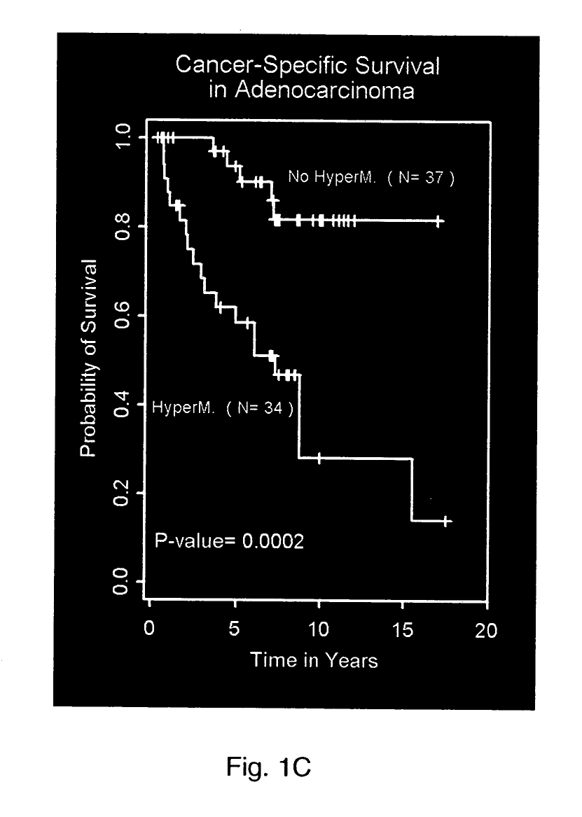

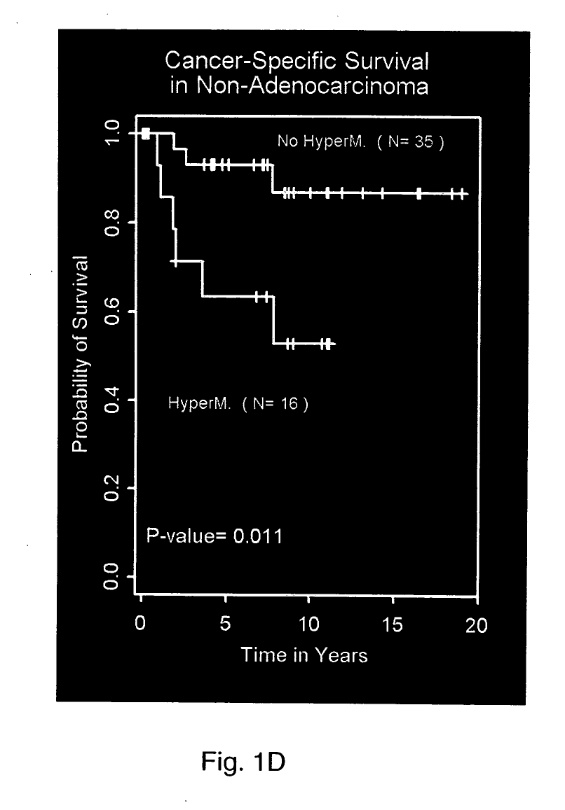

The data were also analyzed for potential associations between the hypermethylation status of the DAP-kinase gene in the primary tumors and patient survival data. Patients whose primary tumors exhibited hypermethylation had a significantly poorer overall survival rate (P=0.041, as assessed using the log-rank test). The probability of survival 5 years after surgery was 68±5% for patients whose tumors did not exhibit hypermethylation, but only 46±7% for patients whose tumor samples exhibited DAP-kinase gene hypermethylation (as indicated in FIG. 1A). Five-year survival rates were significantly different between the non-hypermethylated and hypermethylated groups (P=0.007, as assessed using the Z-test). Survival probability 10 years after surgery was also lower for patients who exhibited a hypermethylated DAP-kinase gene in their tumor DNA.

Strikingly, for the group of patients whose primary tumors did not exhibit hypermethylation at the CpG sites of the DAP-kinase gene, the probability of 5-year disease-specific survival was 92±3%, but only 56±7% for patients in whose tumors DAP-kinase gene hypermethylation occurred (as indicated in FIG. 1B). The probability of 10-year disease-specific survival was similarly strikingly different (83±5% in patients who did not exhibit hypermethylation and 37±8% in those who did). Disease-specific survival rate was highly significantly different between the two groups (P<0.0001, as assessed using the log-rank test and the Z-test). Unlike overall survival, differences in disease-specific survival increased with follow-up time. Similar trends were observed if the 17 patients who died for unknown reasons were included in the disease-specific mortality group.

The data were also assessed in order to detect potential associations between the hypermethylation pattern and disease-specific survival rate in histologic subgroups. Hypermethylation was associated with a poorer disease-specific survival in both adenocarcinoma (P=0.0002) and squamous cell carcinoma (P=0.011), as indicated in FIGS. 1C and 1D.

Multivariate analysis was performed, using the Cox model, in order to determine whether hypermethylation of the CpG sites of the DAP-kinase gene is an independent factor in predicting survival time for patients with pathologic stage I NSCLC. Hypermethylation of the CpG sites in the DAP-kinase gene was found to be the only independent predictor for disease-specific survival rates (P<0.0001) among available parameters, including age, gender, histology, tumor size, and tobacco-smoking/non-smoking status. DAP-kinase hypermethylation was a significant independent factor predicting the overall survival during the first 5 years of follow-up (P=0.008 and P=0.14, respectively).

Many physiological factors such as tumor necrosis factor-alpha, interferon-gamma, and transforming growth factor-beta (TGF-beta) can trigger apoptosis in normal cells (Laster et al., 1988, J. Immunol. 141:2629-2634; Novelli et al., 1994, J. Immunol. 152:496-504; Lin et al., 1992, Cancer Res. 52:385-388). However, tumor cells can lose their ability to respond to these stimulating factors. For example, many lung cancer cell lines do not respond to TGF-beta (Schwarz et al., 1990, Growth Factors 3:115-127), indicating the presence of defects in the TGF-beta-induced signaling pathway.

DAP-kinase was initially identified as a gene whose down-regulation by an anti-sense molecule could prevent HeLa cells from undergoing interferon-gamma-induced apoptosis (Feinstein et al., 1995, Genomics 29:305-307). Others have shown that DAP-kinase is a Ca²⁺/calmodulin-dependent, cytoskeleton-associated protein kinase, and that its apoptosis-inducing function depends on its catalytic activity (Cohen et al., 1997, EMBO J. 16:998-1008). It has been suggested that the ability of DAP-kinase to suppress the metastatic behavior of Lewis lung carcinoma cells in animal models indicates that the protein might function as a metastasis suppressor by inducing apoptosis (Inbal et al., 1997, Nature 390:180-184).

Others studied primary NSCLC samples obtained from 22 patients and observed that DAP-kinase was hypermethylated in 5 (23%) of the patients' tumors (Esteller et al., 1999, Cancer Res. 59:67-70). Although these observations indicate that DAP-kinase hypermethylation is a frequent abnormality in lung cancer patients, those observations do not indicate whether such hypermethylation was an informative indicator of tumorigenesis, tumor progression, or tumor aggressiveness. It was not until the statistically significant studies described in this Example were completed that these associations could be made.

In the studies described in this Example, a panel of 135 tumor samples was assessed in a single clinical stage, which permitted determination of the rate of DAP-kinase hypermethylation across a relatively small subset of patients with lung cancer. 44% of the tumor samples exhibited hypermethylation at the CpG sites of the DAP-kinase gene. Previous studies demonstrated that hypermethylation at the CpG sites of the DAP-kinase can repress expression of the gene (Katzenellenbogen et al., 1999, Blood 93:4347-4353; Kissil et al., 1997, Oncogene 15:403-407). Therefore, using the results described in this Example, it was possible, for the first time, to associate DAP-kinase gene methylation status with tumorigenesis, tumor progression, and tumor aggressiveness.

The results presented in this Example establish that DAP-kinase gene expression can affect one or more of tumorigenesis, tumor progression, and tumor aggressiveness. These results also indicate that tumorigenesis, tumor progression, and tumor aggressiveness can be inhibited by de-methylating a hypermethylated DAP-kinase gene or by inhibiting methylation of this gene.

The most striking finding of the experiments presented in this Example is the strong association observed between DAP-kinase hypermethylation and adverse survival, particularly disease-specific survival. Multivariate analysis indicates that DAP-kinase hypermethylation was the only independent factor for predicting disease-specific survival rates. Several other molecular and genetic markers have been shown to be able to predict outcome of patients with stage I NSCLC, such as loss of heterozygosity, K-ras mutations, and p53 overexpression (Miyake et al., 1999, Oncogene 18:2397-2404; Graziano et al., 1999, J. Clin. Oncol. 17:668-675; Kwiatkowski et al., 1999, J. Clin. Oncol. 16:2468-2477; Rosell et al., 1993, Oncogene 8:2407-2412; Zhou et al., 2000, Clin. Cancer Res. 6:559-565; Herbst et al., 2000, Clin. Cancer Res. 6:790-797). However, contradictory results have also been reported for some markers (Apolinario et al., 1997, J. Clin. Oncol. 15:2456-2466; Pastorino et al., 1997, J. Clin. Oncol. 15:2858-2865), suggesting that the roles of those markers in lung cancer progression are complicated. The results presented in this Example demonstrate for the first time that inactivation of DAP-kinase is an important biomarker for the molecular classification of stage I NSCLC. These findings add one more step towards the development of a model for molecular classification of lung cancer.

The advantages of methylation-specific PCR include the simplicity of the technique, its specificity for the gene, and its high sensitivity. These advantages permit investigators to detect a single altered gene in an environment containing more than 1,000 normal copies of the gene (Herman et al., 1996, Proc. Natl. Acad. Sci. USA 93:9821-9826). In contrast to many other methods of genetic testing, this assay is easy to perform and cost-effective. Furthermore, data interpretation is straightforward, making it possible to compare results across investigators and institutions. It may be that only a small percentage of cells in a particular tumor are capable of metastasis. Therefore, the high sensitivity of methylation-specific PCR will help to identify these abnormal cells among large numbers of cells which do not exhibit this abnormality.

The association between DAP-kinase hypermethylation and poor survival rates indicates that DAP-kinase has an important role in tumor invasion and metastasis of lung cancer. Tumor cells which lack DAP-kinase or which express reduced levels of DAP-kinase demonstrate more aggressive behavior in terms of invasion and metastasis in NSCLC.

Recent data generated by others indicates that the death domain of DAP-kinase is critical in ligand-induced apoptosis (Cohen et al., 1999, J. Cell Biol. 146:141-148). DAP-kinase is also involved in apoptosis induced by tumor necrosis factor-alpha and by Fas. Furthermore, DAP-kinase apoptotic function can be blocked by bcl-2 as well as by p35 inhibitors of caspases (Cohen et al., 1999, J. Cell Biol. 146:141-148). Those observations, in combination with the results presented in this Example, indicate that DAP-kinase is a useful therapeutic target for treatment of NSCLC patients, including those who may harbor a high probability of recurrence and metastasis.

TABLE 1
Demographic characteristics of the patient population

| | Histology | | | |
|---|---|---|---|---|
| | Squamous cell | Adeno- | | |
| | carcinoma | carcinoma | Others | Total |
| # of Patients | 51 (38%) | 71 (53%) | 13 (10%) | 135 (100%) |

Gender

| | | | | |
|---|---|---|---|---|
| Male | 41 (80%) | 48 (68%) | 11 (85%) | 100 (74%) |
| Female | 10 (20%) | 23 (32%) | 2 (15%) | 35 (26%) |
| Mean age (±S.D.) | 64.6 ± 9.1 | 61.3 ± 8.9 | 63.6 ± 8.2 | 62.8 ± 9.0 |

Smoking status

| | | | | |
|---|---|---|---|---|
| Smoker | 43 (81%) | 61 (84%) | 11 (85%) | 115 (85%) |
| Nonsmoker | 8 (19%) | 10 (16%) | 2 (15%) | 20 (15%) |

5-year survival rate in % (±standard error)

| | | | | |
|---|---|---|---|---|
| Overall | 59 ± 7 | 63 ± 6 | 31 ± 13 | 58 ± 4 |
| Disease-specific | 84 ± 6 | 77 ± 5 | 47 ± 15 | 76 ± 4 |

TABLE 2
Hypermethylation of DAP-kinase gene in stage I NSCLC

| Hypermethylation | Yes (%) | No (%) | Total (100%) |
|---|---|---|---|
| Number of patients | 59 (44%) | 76 (56%) | 135 |

Gender^{#}

| | | | |
|---|---|---|---|
| Male | 48 (48%) | 52 (52%) | 100 |
| Female | 11 (31%) | 24 (69%) | 35 |

Age

| | | | |
|---|---|---|---|
| <60 | 21 (40%) | 32 (60%) | 53 |
| ≧60 | 38 (46%) | 44 (54%) | 82 |

Histology*

| | | | |
|---|---|---|---|
| Squamous | 16 (31%) | 35 (69%) | 51 |
| Adeno | 34 (48%) | 37 (52%) | 71 |
| Others | 9 (69%) | 4 (31%) | 13 |

^{#}Test for equal proportion of hypermethylation between male and female, P = .09 (as assessed using the chi-squared test).
*Test for equal proportion of hypermethylation between squamous cell and non-squamous cell tumors, P = .02 (as assessed using the chi-squared test). When the equal proportion of hypermethylation between squamous cell carcinoma and adenocarcinoma was tested, P equals to .067 (as assessed using the chi-squared test).

### Example 2

The HOXA9 Gene is Widely Activated in Bronchial Epithelium of Patients Afflicted With Lung Cancer

Homeobox (HOX) genes have an important role in pattern formation during development and in maintaining the differentiated state of cells in an adult organism (Krumlauf, 1994, Cell 78:191-201; Vincent et al., 1994, Cell 77:909-915). Deregulation of HOX genes has an important role in tumorigenesis. For example, t(10;14)(q24;q11) translocation was detected in a subset of T-cell leukemia cells and activated HOX11 (Hatano et al., 1991, Science 253:79-82). Similarly, HOXA9 is transcriptionally activated in a subset of acute myeloid leukemias when the t(7;11)(p15;p15) translocation occurs (Nakamura et al., 1996, Nature Genet. 12:154-158). Activation of the HOXB3, HOXB4, and HOXC6 genes in lung carcinomas has also been reported (Bodey et al., 2000, Anticancer Res. 20:2711-2716).

A survey was performed in order to detect deregulation of HOX genes in lung cancer-associated cells. A panel of NSCLC cell lines was examined, and it was determined that the HOXA9 gene was expressed in all cell lines analyzed, as assessed using reverse transcription polymerase chain reaction (RT-PCR). HOXA9 gene expression could not detected in this way in a cDNA library generated from the lung tissue of a 17-year-old female non-smoker or in cDNA generated from a normal bronchial epithelial cell line transformed with the SV-40 large antigen.

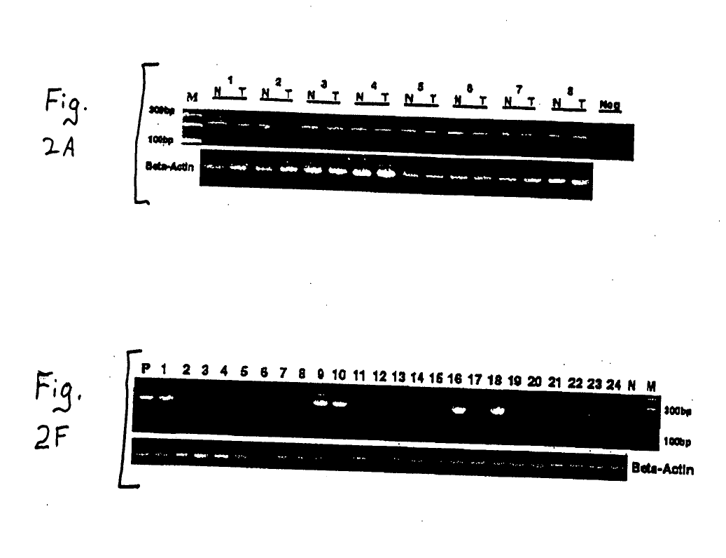

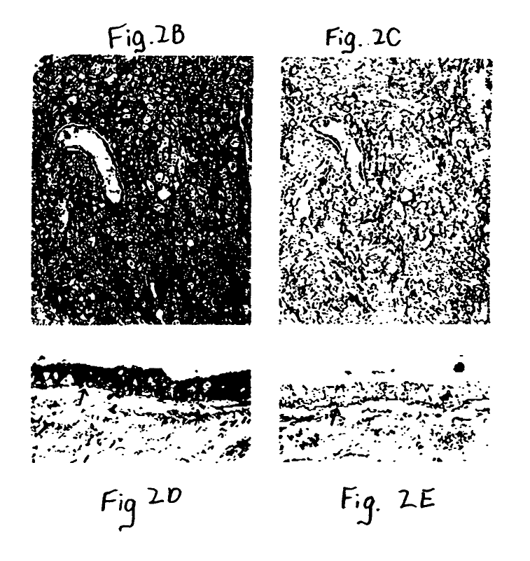

Surgically resected primary NSCLC tumors obtained from 30 patients were assessed, and it was determined that 27 (90%) of the 30 tumors expressed HOXA9 messenger RNA (mRNA; as illustrated in FIG. 2A). PCR primers used in the HOXA9 detection methods were designed to flank a 1-kb intron to amplify a 218-bp cDNA fragment. The sequences of these primers were CCGGCCTTAT GGCATTAAAC (SEQ ID NO: 1) and AGTTGGCTGC TGGGTTATTG (SEQ ID NO: 2). Thus, PCR amplification products generated from contaminating genomic DNA could be easily distinguished from those generated from cDNA, owing to the size differences attributable to the presence (i.e., in genomic DNA) or absence (i.e., in the cDNA) of the intron. The RT-PCR amplification product having the expected size was directly sequenced, and matched the published HOXA9 mRNA sequence. Surprisingly, HOXA9 was expressed not only in NSCLC cells, but also in corresponding normal lung tissues located distant to the primary NSCLC in all 30 tumors, suggesting that HOXA9 is activated and has an important role in the early development of NSCLC.

In order to assess local HOXA9 expression at the cellular level, mRNA in situ hybridization was performed using an antisense ribonucleotide probe that specifically hybridized with HOXA9 mRNA. The nucleotide sequence of this probe was CCGGCCTTAT GGCATTAAAC CTGAACCGCT GTCGGCCAGA AGGGGTGACT GTCCCACGCT TGACACTCAC ACTTTGTCCC TGACTGACTA TGCTTGTGGT TCTCCTCCAG TTGATAGAGA AAAACAACCC AGCGAAGGCG CCTTCTCCGA AAACAATGCC GAGAATGAGA GCGGCGGAGA CAAGCCCCCC ATCGATCCCA ATAACCCAGC AGCCAACT (SEQ ID NO: 3). Expression of HOXA9 was found to be restricted to lung carcinoma cells and bronchial epithelial cells in the corresponding normal lung tissues in all 5 pairs of tumor/normal tissue pairs analyzed, as illustrated in FIGS. 2B-2E).

In order to determine whether expression of the HOXA9 gene in normal bronchial epithelium precedes development of invasive lung cancer (i.e., rather than merely being symptomatic of NSCLC) bronchial brush tissue specimens obtained from former smokers were analyzed for HOXA9 expression. Although none of these individuals exhibited symptoms of lung cancer, they have a high risk to develop lung cancer. HOXA9 expression was detected in 5 (21%) of the 24 specimens analyzed, as illustrated in FIG. 2F. The frequencies of HOXA9 expression in epithelial cells obtained from patients afflicted with NSCLC (frequency=100%) and those obtained from former smokers (frequency=24%) are statistically significant (P>0.001, as assessed using Fisher's exact test). These results indicate that activation of HOXA9 in bronchial cells is an early step necessary for the development of NSCLC.

HOXA9 expression can therefore be used as a biomarker for identification of high-risk population or for diagnosis of lung cancer at an early stage, either alone or in combination with other strategies such as spiral computer tomography (Henschke et al., 1999, Lancet 354:99-105). These results also indicate that tumorigenesis and tumor progression associated with NSCLC require HOXA9 gene expression. Thus, compounds which inhibit expression of the HOXA9 gene can be used to inhibit or reverse tumorigenesis and tumor progression in lung cells. By assaying cells which normally express (or which have been caused to express) the HOXA9 gene in the presence and absence of a test compound, one can determine whether the test compound is useful for preventing, inhibiting, treating, or even curing NSCLC.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1-26. (canceled)

27. A method of diagnosing non-small cell lung cancer (NSCLC) in a human, the method comprising assessing expression of the HOXA9 gene in lung cells of the human, whereby a greater degree of expression of the gene in the human relative to a normal level of expression of the gene in humans not afflicted with NSCLC is an indication that the human is afflicted with NSCLC.

28. The method of claim 27 wherein expression of the gene is assessed in vitro in cells obtained from the human.

29. The method of claim 28 wherein the cells are obtained from a bronchial lavage.

30. The method of claim 28 wherein the cells are epithelial cells.

31. The method of claim 27 wherein the human does not exhibit a macroscopic clinical symptom of NSCLC.

32. The method of claim 31 wherein the symptom is selected from the group consisting of a persistent cough which gets worse over time, constant chest pain, coughing up blood, shortness of breath, wheezing, hoarseness, recurrent pneumonia, recurrent bronchitis, swelling of the neck and face, loss of appetite, weight loss, and fatigue.

33. The method of claim 27 wherein expression of the gene is assessed using a first oligonucleotide which specifically hybridizes with a transcription product of the gene.

34. The method of claim 33 wherein the first oligonucleotide does not specifically hybridize with the gene.

35. The method of claim 33 wherein the first oligonucleotide and a second oligonucleotide are used in a polymerase chain reaction (PCR) to amplify a portion of the gene.

36. The method of claim 35 wherein the portion includes sub-portions wherein an intron is interposed between the sub-portions of the gene, but wherein the sub-portions are adjacent in mRNA derived from the gene.

37. A method of assessing the risk that a human will develop NSCLC, the method comprising assessing expression of the HOXA9 gene in lung cells of the human, whereby a greater degree of expression of the gene in the human relative to a normal level of expression of the gene in humans not afflicted with NSCLC is an indication that the human is at an increased risk for developing NSCLC.

38-42. (canceled)


SEQUENCE LISTING (7 sequences)

SEQ ID NO: 1 - DNA, 20 bp, Artificial
  HoxA9 PCR Primer
ccggccttatggcattaaac

SEQ ID NO: 2 - DNA, 20 bp, Artificial
  HoxA9 PCR Primer
agttggctgctgggttattg

SEQ ID NO: 3 - DNA, 218 bp, Artificial
  HoxA9 Probe
ccggccttatggcattaaacctgaaccgctgtcggccagaaggggtgactgtcccacgcttgacactcacactttgtccctgactgactatgcttgtggttctcctccagttgatagagaaaaacaacccagcgaaggcgccttctccgaaaacaatgccgagaatgagagcggcggagacaagccccccatcgatcccaataacccagcagccaact

SEQ ID NO: 4 - DNA, 5910 bp, Homo sapiens
  CDS (337)..(4632)
cggaggacagccggaccgagccaacgccggggactttgttccctccacggaggggactcggcaactcgcagcggcagggtctggggccggcgcctgggagggatctgcgccccccactcactccctagctgtgttcccgccgccgccccggctagtctccggcgctggcgcctatggtcggcctccgacagcgctccggagggaccgggggagctcccaggcgcccgggactggagactgatgcatgaggggcctacggaggcgcaggagcggtggtgatggtctgggaagcggagctgaagtcccctgggctttggtgaggcgtgacagtttatcatgaccgtgttcaggcaggaaaacgtggatgattactacgacaccggcgaggaacttggcagtggacagtttgcggttgtgaagaaatgccgtgagaaaagtaccggcctccagtatgccgccaaattcatcaagaaaaggaggactaagtccagccggcggggtgtgagccgcgaggacatcgagcgggaggtcagcatcctgaaggagatccagcaccccaatgtcatcaccctgcacgaggtctatgagaacaagacggacgtcatcctgatcttggaactcgttgcaggtggcgagctgtttgacttcttagctgaaaaggaatctttaactgaagaggaagcaactgaatttctcaaacaaattcttaatggtgtttactacctgcactcccttcaaatcgcccactttgatcttaagcctgagaacataatgcttttggatagaaatgtccccaaacctcggatcaagatcattgactttgggttggcccataaaattgactttggaaatgaatttaaaaacatatttgggactccagagtttgtcgctcctgagatagtcaactatgaacctcttggtcttgaggcagatatgtggagtatcggggtaataacctatatcctcctaagtggggcctccccatttcttggagacactaagcaagaaacgttagcaaatgtatccgctgtcaactacgaatttgaggatgaatacttcagtaataccagtgccctagccaaagatttcataagaagacttctggtcaaggatccaaagaagagaatgacaattcaagatagtttgcagcatccctggatcaagcctaaagatacacaacaggcacttagtagaaaagcatcagcagtaaacatggagaaattcaagaagtttgcagcccggaaaaaatggaaacaatccgttcgcttgatatcactgtgccaaagattatccaggtcattcctgtccagaagtaacatgagtgttgccagaagcgatgatactctggatgaggaagactcctttgtgatgaaagccatcatccatgccatcaacgatgacaatgtcccaggcctgcagcaccttctgggctcattatccaactatgatgttaaccaacccaacaagcacgggacacctccattactcattgctgctggctgtgggaatattcaaatactacagttgctcattaaaagaggctcgagaatcgatgtccaggataagggcgggtccaatgccgtctactgggctgctcggcatggccacgtcgataccttgaaatttctcagtgagaacaaatgccctttggatgtgaaagacaagtctggagagatggccctccacgtggcagctcgctatggccatgctgacgtggctcaagttacttgtgcagcttcggctcaaatcccaatatccaggacaaaggaagaagaaacccccctgcactgtgctgcttggcacggctattactctgtggccaaagccctttgtgaagccggctgtaacgtgaacatcaagaaccgagaaggagagacgcccctcctgacagcctctgccaggggctaccacgacatcgtggagtgtctggccgaacatggagccgaccttaatgcttgcgacaaggacggacacattgcccttcatctggctgtaagacggtgtcagatggaggtaatcaagactctcctcagccaagggtgtttcgtcgattatcaagacaggcacggcaatactcccctccatgtggcatgtaaagatggcaacatgcctatcgtggtggccctctgtgaagcaaactgcaatttggacatctccaacaagtatgggcgaacgcctctgcaccttgcggccaacaacggaatcctagacgtggtccggtatctctgtctgatgggagccagcgttgaggcgctgaccacggacggaaagacggcagaagatcttgctagatcggaacagcacgagcacgtagcaggtctccttgcaagacttcgaaaggatacgcaccgaggactcttcatccagcagctccgacccacacagaacctgcagccaagaattaagctcaagctgtttggccactcgggatccgggaaaaccacccttgtagaatctctcaagtgtgggctgctgaggagctttttcagaaggcgtcggcccagactgtcttccaccaactccagcaggttcccaccttcacccctggcttctaagcccacagtctcagtgagcatcaacaacctgtacccaggctgcgagaacgtgagtgtgaggagccgcagcatgatgttcgagccgggtcttaccaaagggatgctggaggtgtttgtggccccgacccaccacccgcactgctcggccgatgaccagtccaccaaggccatcgacatccagaacgcttatttgaatggagttggcgatttcagcgtgtgggagttctctggaaatcctgtgtatttctgctgttatgactattttgctgcaaatgatcccacgtcaatccatgttgttgtctttagtctagaagagccctatgagatccagctgaacccagtgattttctggctcagtttcctgaagtcccttgtcccagttgaagaacccatagccttcggtggcaagctgaagaacccactccaagttgtcctggtggccacccacgctgacatcatgaatgttcctcgaccggctggaggcgagtttggatatgacaaagacacatcgttgctgaaagagattaggaacaggtttggaaatgatcttcacatttcaaataagctgtttgttctggatgctggggcttctgggtcaaaggacatgaaggtacttcgaaatcatctgcaagaaatacgaagccagattgtttcggtctgtcctcccatgactcacctgtgtgagaaaatcatctccacgctgccttcctggaggaagctcaatggacccaaccagctgatgtcgctgcagcagtttgtgtacgacgtgcaggaccagctgaaccccctggccagcgaggaggacctcaggcgcattgctcagcagctccacagcacaggcgagatcaacatcatgcaaagtgaaacagttcaggacgtgctgctcctggacccccgctggctctgcacaaacgtcctggggaagttgctgtccgtggagaccccacgggcgctgcaccactaccggggccgctacaccgtggaggacatccagcgcctggtgcccgacagcgacgtggaggagctgctgcagatcctcgatgccatggacatctgcgcccgggacctgagcagcgggaccatggtggacgtcccagccctgatcaagacagacaacctgcaccgctcctgggctgatgaggaggacgaggtgatggtgtatggtggcgtgcgcatcgtgcccgtggaacacctcacccccttcccatgtggcatctttcacaaggtccaggtgaacctgtgccggtggatccaccagcaaagcacagagggcgacgcggacatccgcctgtgggtgaatggctgcaagctggccaaccgtggggccgagctgctggtgctgctggtcaaccacggccagggcattgaggtccaggtccgtggcctggagacggagaagatcaagtgctgcctgctgctggactcggtgtgcagcaccattgagaacgtcatggccaccacgctgccagggctcctgaccgtgaagcattacctgagcccccagcagctgcgggagcaccatgagcccgtcatgatctaccagccacgggacttcttccgggcacagactctgaaggaaacctcactgaccaacaccatgggggggtacaaggaaagcttcagcagcatcatgtgcttcgggtgtcacgacgtctactcacaggccagcctcggcatggacatccatgcatcagacctgaacctcctcactcggaggaaactgagtcgcctgctggacccgcccgaccccctggggaaggactggtgccttctcgccatgaacttaggcctccctgacctcgtggcaaagtacaacaccaataacggggctcccaaggatttcctccccagccccctccacgccctgctgcgggaatggaccacctaccctgagagcacagtgggcaccctcatgtccaaactgagggagctgggtcgccgggatgccgcagaccttttgctgaaggcatcctctgtgttcaaaatcaacctggatggcaatggccaggaggcctatgcctcgagctgcaacagcggcacctcttacaattccattagctctgttgtatcccggtgagggcagcctctggcttggacagggtctgtttggactgcagaaccaagggggtgatgtagcccatccttccctttggagatgctgagggtgtttcttcctgcacccacagccagggggatgccactcctccctccggcttgacctgtttctctgccgctacctccctccccgtctcattccgttgtctgtggatggtcattgcagtttaagagcagaacagatcttttactttggccgcttgaaaagctagtgtacctcctctcagtgttttggactccatctctcatcctccagtaccttgcttcttactgataattttgctggaattcctaacttttcaatgacattttttttaactatcatattgattgtcctttaaaaaagaaaagtgcatatttatccaaaatgtgtatttcttatacgcttttctgtgttataccatttcctcagcttatctcttttatatttgtaggagaaactcccatgtatggaatcccactgtatgatttataaacagacaatatgtgagtgccttttgcagaagagggtgtgtttgaaatcatcggagtcagccaggagctgtcaccaaggaaacgctacctctctgtcccttgctgtatgctgatcatcgccagaggtgcttcaccctgagttttgttttgtattgttttctgacagtttttctgttttgtttggcaaggaaaggggagaagggaatcctcctccagggtgattttatgatcagtgttgttgctctaggaagacatttttccgtttgcttttgttccaatgtcaatgtgaacgtccacatgaaacctacacactgtcatgcttcatcattccctctcatctcaggtagaaggttgacacagttgtagggttacagagacctatgtaagaattcagaagacccctgactcatcatttgtggcagtcccttataattggtgcatagcagatggtttccacatttagatcctggtttcataacttcctgtacttgaagtctaaaagcagaaaataaaggaagcaagttttcttccatgattttaaattgtgatcgagttttaaattgataggagggaacatgtcctaattcttctgtcctgagaagcatgtaatgttaatgttatatcatatgtatatatatatatgcactatgtatatacatatatattaatactggtatttttacttaatctataaaatgtcgttaaaaagttgtttgtttttttctttttttataaataaactgttgctcgttaaaaaaaaaaaaaa

SEQ ID NO: 5 - protein, 1431 aa, Homo sapiens
MTVFRQENVDDYYDTGEELGSGQFAVVKKCREKSTGLQYAAKFIKKRRTKSSRRGVSREDIEREVSILKEIQHPNVITLHEVYENKTDVILILELVAGGELFDFLAEKESLTEEEATEFLKQILNGVYYLHSLQIAHFDLKPENIMLLDRNVPKPRIKIIDFGLAHKIDFGNEFKNIFGTPEFVAPEIVNYEPLGLEADMWSIGVITYILLSGASPFLGDTKQETLANVSAVNYEFEDEYFSNTSALAKDFIRRLLVKDPKKRMTIQDSLQHPWIKPKDTQQALSRKASAVNMEKFKKFAARKKWKQSVRLISLCQRLSRSFLSRSNMSVARSDDTLDEEDSFVMKAIIHAINDDNVPGLQHLLGSLSNYDVNQPNKHGTPPLLIAAGCGNIQILQLLIKRGSRIDVQDKGGSNAVYWAARHGHVDTLKFLSENKCPLDVKDKSGEMALHVAARYGHADVAQVTCAASAQIPISRTKEEETPLHCAAWHGYYSVAKALCEAGCNVNIKNREGETPLLTASARGYHDIVECLAEHGADLNACDKDGHIALHLAVRRCQMEVIKTLLSQGCFVDYQDRHGNTPLHVACKDGNMPIVVALCEANCNLDISNKYGRTPLHLAANNGILDVVRYLCLMGASVEALTTDGKTAEDLARSEQHEHVAGLLARLRKDTHRGLFIQQLRPTQNLQPRIKLKLFGHSGSGKTTLVESLKCGLLRSFFRRRRPRLSSTNSSRFPPSPLASKPTVSVSINNLYPGCENVSVRSRSMMFEPGLTKGMLEVFVAPTHHPHCSADDQSTKAIDIQNAYLNGVGDFSVWEFSGNPVYFCCYDYFAANDPTSIHVVVFSLEEPYEIQLNPVIFWLSFLKSLVPVEEPIAFGGKLKNPLQVVLVATHADIMNVPRPAGGEFGYDKDTSLLKEIRNRFGNDLHISNKLFVLDAGASGSKDMKVLRNHLQEIRSQIVSVCPPMTHLCEKIISTLPSWRKLNGPNQLMSLQQFVYDVQDQLNPLASEEDLRRIAQQLHSTGEINIMQSETVQDVLLLDPRWLCTNVLGKLLSVETPRALHHYRGRYTVEDIQRLVPDSDVEELLQILDAMDICARDLSSGTMVDVPALIKTDNLHRSWADEEDEVMVYGGVRIVPVEHLTPFPCGIFHKVQVNLCRWIHQQSTEGDADIRLWVNGCKLANRGAELLVLLVNHGQGIEVQVRGLETEKIKCCLLLDSVCSTIENVMATTLPGLLTVKHYLSPQQLREHHEPVMIYQPRDFFRAQTLKETSLTNTMGGYKESFSSIMCFGCHDVYSQASLGMDIHASDLNLLTRRKLSRLLDPPDPLGKDWCLLAMNLGLPDLVAKYNTNNGAPKDFLPSPLHALLREWTTYPESTVGTLMSKLRELGRRDAADLLLKASSVFKINLDGNGQEAYASSCNSGTSYNSISSVVSR

SEQ ID NO: 6 - DNA, 597 bp, Homo sapiens
  CDS (1)..(597)
atggcagggttctctccttggcggcggcggcagcggcggaggcggcggcggcggcgggcgaggcacgcttcgcgggcagcaccagaactggtcggtgatttaggtagtttcctgttgttgggatccacctttctctcgacaggcacgacactgcccttcattacttcagttgaaatcgtctccaggtacctctgcgcgcgggggtcgggccgcgcggggcatcacggccctggtcgtgccaggcctgcggtggcaacctcggctttccctgctcaggagcctcgtgtctttctccgcagcgctttgccagccggccggctttccccttccaccacacacctccacctggtcacagcagataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtga

SEQ ID NO: 7 - protein, 198 aa, Homo sapiens
MAGFSPWRRRQRRRRRRRRARHASRAAPELVGDLGSFLLLGSTFLSTGTTLPFITSVEIVSRYLCARGSGRAGHHGPGRARPAVATSAFPAQEPRVFLRSALPAGRLSPSTTHLHLVTADNPAANWLHARSTRKKRCPYTKHQTLELEKEFLFNMYLTRDRRYEVARLLNLTERQVKIWFQNRRMKMKKINKDRAKDE